(19) United States
(12) Patent Application Publication
(10) Pub. No.: US 2018/0043118 A1
(43) Pub. Date: Feb. 15, 2018

(54) TRACHEOSTOMY TUBE ASSEMBLIES

(71) Applicant: SMITHS MEDICAL INTERNATIONAL LIMITED, Ashford (GB)

(72) Inventors: Andrew Thomas Jeffrey, Hythe (GB); Christopher John Woosnam, Great Sutton (GB)

(73) Assignee: SMITHS MEDICAL INTERNATIONAL LIMITED, Ashford (GB)

(21) Appl. No.: 15/558,216

(22) PCT Filed: Feb. 15, 2016
(86) PCT No.: PCT/GB2016/000031

§ 371 Date: Sep. 14, 2017

(30) Foreign Application Priority Data
Mar. 21, 2015 (GB) 1504914.1

(51) Int. Cl. A61M 16/04; A61M 16/08

(52) U.S. Cl. CPC A61M 16/0465; A61M 16/0825; A61M 16/0497

(57) ABSTRACT

A tracheostomy tube has a ball formation (31) of part-spherical shape at its machine end (13). The ball formation (31) is retained between two rings (33) and (34) projecting rearwardly from the tube neck flange (20) to enable movement between the tube shaft (10) and the flange in multiple planes.

This invention relates to tracheostomy tube assemblies of the kind including a shaft having a patient end adapted for location within the trachea and a machine end adapted for location outside the patient, the assembly including a flange adapted to lie against the neck surface and a coupling by which the flange is attached with the shaft.

Tracheostomy tubes are used to enable ventilation or respiration of a patient. The tube is inserted into the trachea via a surgically-formed opening in the neck so that one end locates in the trachea and the other end locates outside the patient adjacent the neck surface. Various types of different tracheostomy tubes are presently available to suit different needs. Tracheostomy tubes are also available with an inner cannula, which can be removed and replaced periodically to prevent the build-up of secretions and avoid the need to replace the tube itself Tracheostomy tubes can be inserted by different techniques, such as the surgical cut down procedure carried out in an operating theatre or a percutaneous dilatation procedure, which may be carried out in emergency situations.

Tracheostomy tubes are generally used for more long-term ventilation or where it is not possible to insert an airway through the mouth or nose. The patient is often conscious while breathing through a tracheostomy tube, which may be open to atmosphere or connected by tubing to some form of ventilator. The tube is secured in position by means of a flange fixed with the machine end of the tube that is positioned to extend outwardly on opposite sides of the tube. The flange has apertures on either side to which a neck strap can be fastened. There can be considerable differences in the anatomy between different patients, which may make it difficult to secure the flange with the neck surface without putting excessive force on the tube, even though the flange and its attachment with the tube may be flexible. Force applied to the tube in this way can lead to incorrect positioning of the tube in the trachea, can cause trauma to patient tissue in or around the stoma and can cause discomfort to the patient. Discomfort can also be caused to the patient when he moves because of the relatively inflexible connection between the flange and shaft of conventional tubes.

It is an object of the present invention to provide an alternative tracheostomy tube assembly.

According to the present invention there is provided a tracheostomy tube assembly of the above-specified kind, characterised in that the coupling includes a part-spherical surface on the shaft and a cooperating formation on the flange adapted to engage with the part-spherical surface on the shaft such as to allow angular movement of the flange relative to the shaft.

The cooperating formation preferably includes two ring members projecting from the flange on opposite sides of the part-spherical surface such that the part-spherical surface is retained by engagement in respective circular apertures in the ring members. The ring members are preferably arranged at right angles to the length of the flange. The shaft preferably includes a connector hub projecting from the machine side of the part-spherical surface. The part-spherical surface preferably extends around the entire circumference of the shaft and extends for an angle of substantially 90° around an axis orthogonal to the plane of the shaft.

A tracheostomy tube assembly according to the present invention will now be described, by way of example, with reference to the accompanying drawings, in which:

FIG. 1 is a perspective view of the tracheostomy tube assembly;

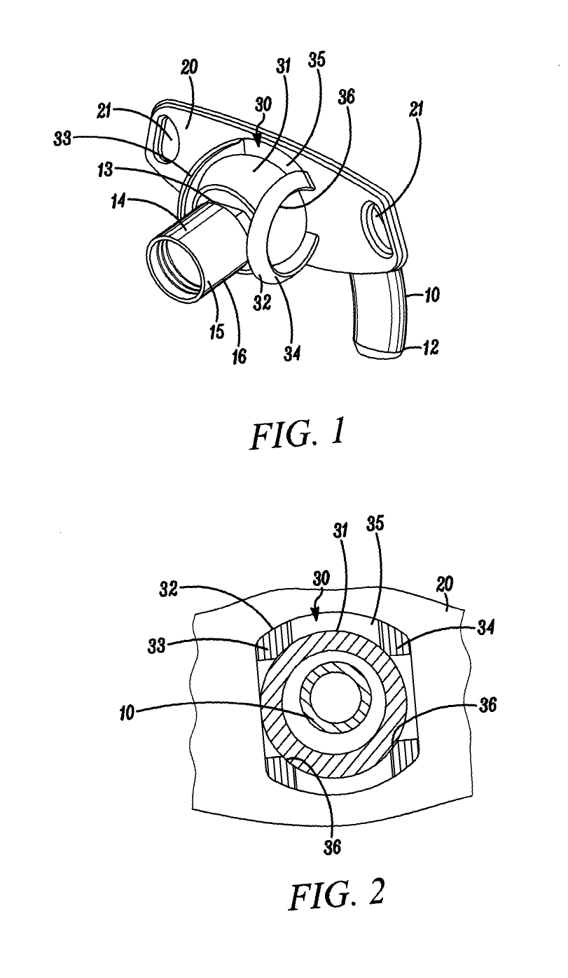

FIG. 2 is a cross-sectional view of the assembly in the region of the coupling;

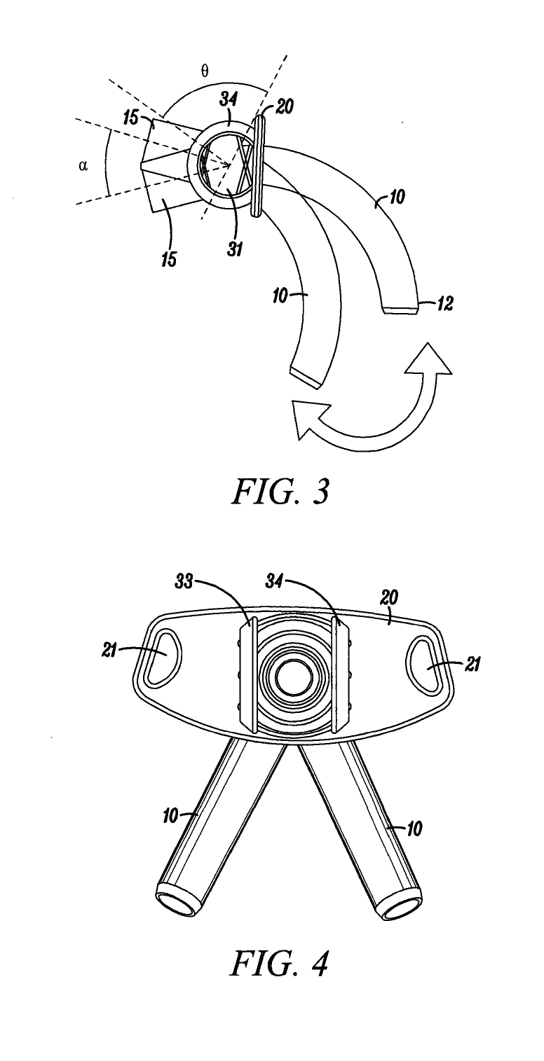

FIG. 3 is a side elevation view of the tracheostomy tube assembly showing the extremes of displacement of the tube shaft in a vertical plane along the axis of the shaft; and

FIG. 4 is an end view of the tracheostomy tube assembly from the machine end showing the extremes of displacement of the tube shaft about a horizontal axis aligned with the machine end of the shaft.

With reference first to FIGS. 1 and 2, the tracheostomy assembly comprises a curved shaft 10 of circular section with a patient end 12 adapted to be located within the trachea of the patient. The shaft 10 may have a conventional sealing cuff towards its patient end although the tube shown does not have any sealing cuff. The shaft 10 is moulded or extruded and is bendable but relatively stiff; being made of a plastics material such as PVC or silicone. The machine end 13 of the shaft 10 is adapted, during use to be located externally and adjacent the tracheostomy opening formed in the patient's neck. The machine end 13 of the shaft 10 supports a hub 14 and a 15 mm male tapered connector 15 fixed with or forming a part of the hub and having a tapered external surface 16. The connector 15 is adapted to make a removable push fit in a conventional 15 mm female connector (not shown) at one end of a breathing tube extending to a ventilator or anaesthetic machine.

The tracheostomy tube assembly also includes a radially-extending support flange 20 that is adapted to lie against the skin surface of the neck on either side of the tracheostomy stoma. The flange 20 has openings 21 at opposite ends for attachment to a neck strap (not shown) used to support the tube with the patient's neck.

The flange 20 is attached with the shaft 10 by coupling means in the form of a ball and socket arrangement 30. The ball and socket arrangement 30 includes a part-spherical ball enlargement 31 at the patient end of the hub 14 so that the hub projects from the machine side of the ball enlargement. The enlargement 31 extends around the entire circumference of the shaft 10 and hub 14 and extends through an angle θ (FIG. 3) of about 90° around an axis orthogonal to the plane of the shaft 10 and hub 14. The diameter of the spherical enlargement 31 is about 25% greater than the diameter of the outside of the hub 14. The cooperating formation or socket part 32 of the arrangement 30 is provided by two rings 33 and 34 moulded integrally with the flange 20 and projecting at right angles rearwardly from the flange on either side of a central aperture 35 through which the shaft 10 extends. Each ring 33 and 34 has a central, circular aperture 36 with a curved surface having a radius matched with the external diameter of the ball enlargement 31. The diameter of the ball enlargement 31 and the dimensions and spacing of the two rings 33 and 34 of the cooperating socket formation 32 are such that the ball enlargement is gripped securely between the rings but allow sliding movement between them. This sliding movement is enhanced by suitable selection of the plastics materials from which the ball enlargement 31 and the rings 33 and 34 are formed, to enable a relatively low friction displacement. The rings 33 and 34 are shown as being oriented at right angles to the length of the flange 20, that is, generally parallel to the axis of the shaft 10. However, it would be possible to locate the rings at the top and bottom of the flange so that they are aligned, instead, parallel to the length of the flange 20.

FIGS. 3 and 4 illustrate the extent of displacement between the flange 20 and the shaft 10 that is made possible by the ball and socket arrangement 30. More particularly, as shown in FIG. 3, the shaft 10 can be displaced through an angle a of about 45° in a plane including the axis of the shaft, when the flange 20 is in a fixed position. FIG. 4 shows that the shaft 10 can also be displaced through a full 360° about its axis at its machine end 13 although, in use, this angle will be substantially limited by the patient's anatomy. Depending on the spacing between the rings 33 and 34 and the hub 14 it may also be possible to displace the shaft 10 about a vertical axis parallel to the plane of the flange 20 and passing through the centre of curvature of the ball enlargement 31.

In use, therefore, the flange 20 can be positioned flat against the surface of the neck regardless of small anatomical variations between patients, because the shaft 10 can articulate up or down to adopt the correct alignment with the stoma and the trachea with least force on patient tissue. The assembly also overcomes the problem of the discomfort or damage that patients with a tracheostomy tube can suffer when changing position, such as when turning their head or moving in bed. Although the neck flange 20 is secured firmly in position, the tube shaft 10 can articulate to accommodate changes in angle to position itself and fit more naturally in the trachea. The assembly can also accommodate patients with an unusual anatomy and abnormal trachea.

1-5. (canceled)

6. A tracheostomy tube assembly including a shaft having a patient end adapted for location within the trachea and a machine end adapted for location outside the patient, the assembly including a flange adapted to lie against the neck surface and a coupling by which the flange is attached with the shaft, characterised in that the coupling includes a part-spherical surface on the shaft and a cooperating formation on the flange adapted to engage with the part-spherical surface on the shaft such as to allow angular movement of the flange relative to the shaft.

7. A tracheostomy tube assembly according to claim 6, characterised in that the cooperating formation includes two ring members projecting from the flange on opposite sides of the part-spherical surface such that the part-spherical surface is retained by engagement in respective circular apertures in the ring members.

8. A tracheostomy tube assembly according to claim 7, characterised in that the ring members are arranged at right angles to the length of the flange.

9. A tracheostomy tube assembly according to claim 6, characterised in that the shaft includes a connector hub projecting from the machine side of the part-spherical surface.

10. A tracheostomy tube assembly according to claim 6, characterised in that the part-spherical surface extends around the entire circumference of the shaft and extends through an angle of substantially 90° around an axis orthogonal to the plane of the shaft.
